(19) United States
(12) Patent Application Publication
(10) Pub. No.: US 2026/0232229 A1
(43) Pub. Date: Aug. 13, 2026

(54) CORRECTING TEMPORAL BIAS IN TUMOR MEASUREMENTS USING TUMOR GROWTH KINETICS

(71) Applicant: IMAGING ENDPOINTS II LLC, Scottsdale, AZ (US)

(72) Inventors: Andre Burkett, Sunny Isles Beach, FL (US); Dominic Zygadlo, Scottsdale, AZ (US); Ronald Lee Korn, Paradise Valley, AZ (US)

(21) Appl. No.: 19/536,359

(22) Filed: Feb. 11, 2026

Related U.S. Application Data
(60) Provisional application No. 63/757,241, filed on Feb. 11, 2025.

(51) Int. Cl. A61B 5/145

(52) U.S. Cl. CPC A61B 5/14532

(57) ABSTRACT

Temporal bias in measurements of tumor burden based on medical image data is corrected. Medical image data, which include medical images acquired at multiple timepoints for each subject in a study, is received with a computer system. Tumor burden measurement data are generated from the medical image data. The tumor burden measurement data indicate a tumor burden estimate for each of the timepoints for each of the subjects. A tumor growth kinetics (TGK) model is computed for each of the subjects based on the tumor burden measurement data generated for the respective one of the subjects. A temporal bias is estimated for each of the subjects based on the respective TGK model, and corrected tumor burden measurement data are generated using the estimated temporal bias.

### CROSS-REFERENCE TO RELATED APPLICATIONS

This application claims the benefit of U.S. Provisional Patent Application Ser. No. 63/757,241, filed on Feb. 11, 2025, and entitled “Correcting Temporal Bias in Tumor Measurements Using Tumor Growth Kinetics,” which is herein incorporated by reference in its entirety.

### BACKGROUND

It is often desirable, for researchers in the field of radiology to aggregate large amounts of data from multiple sources for analysis to determine the effectiveness of a cancer treatment. Unfortunately, the timing of image acquisition is often unstandardized, particularly, outside of the clinical trial setting. Even in clinical trial settings, patients often have imaging performed outside of their protocol-defined imaging windows for a variety of reasons. This variability, along with the fact that tumors change in size over time, can make it very difficult for researchers to draw unbiased conclusions from their analyses.

Currently, there is no industry accepted methodology to measure the bias created from non-uniform image acquisition times, nor is there a method to normalize the data to reduce this bias.

### SUMMARY OF THE DISCLOSURE

The present disclosure addresses the aforementioned drawbacks by providing a method for correcting temporal bias in measurements of tumor burden based on medical image data. The method includes receiving medical image data with a computer system, where the medical image data comprise medical images acquired at a plurality of timepoints for each of a plurality of subjects. Tumor burden measurement data are generated from the medical image data using the computer system, where the tumor burden measurement data indicate a tumor burden estimate for each of the plurality of timepoints for each of the plurality of subject. A tumor growth kinetics (TGK) model is computed for each of the plurality of subjects based on the tumor burden measurement data generated for the respective one of the plurality of subjects. A temporal bias is estimated for each of the plurality of subjects based on the TGK model for the respective one of the plurality of subjects. Corrected tumor burden measurement data are generated using the estimated temporal bias.

### BRIEF DESCRIPTION OF THE DRAWINGS

FIGS. 1A and 1B illustrate a flowchart of an example method for evaluating and

correcting for temporal measurement bias in tumor burden measurement data.

FIG. 2 illustrates an example timepoint days from start (DFS) distribution ridgeline plot.

FIG. 3 illustrates an example timepoint subgroup DFS distribution ridgeline plot FIG. 4 illustrates an example response count comparison line plot.

FIG. 5 illustrates an example response count subgroup comparison line plot.

FIG. 6 illustrates an example partial response (PR)/progressive disease (PD) response ratio line plot.

FIG. 7 illustrates an example PR/PD subgroup response ratio line plot

FIG. 8 illustrates an example significance of adjusted response measurements from timepoint center of window (TCW) plot.

FIG. 9 illustrates an example relative bias between adjusted sum of diameter (SOD) measurements and median day plot.

FIG. 10 is a block diagram of an example system for correcting or otherwise reducing temporal measurement bias in tumor burden measurement data.

FIG. 11 is a block diagram of example components that can implement the system of FIG. 10.

### DETAILED DESCRIPTION

Described here are systems and methods for measuring and reducing temporal measurement bias (TMB) in tumor measurements using tumor growth kinetics (TGK), such as exponential TGK (eTGK). As a non-limiting example, the disclosed systems and methods utilize adjusted tumor burden (ATB) and adjusted response categorizations (ARCs), which allow for the estimation of each patient's tumor burden and response to treatment at a common point in time.

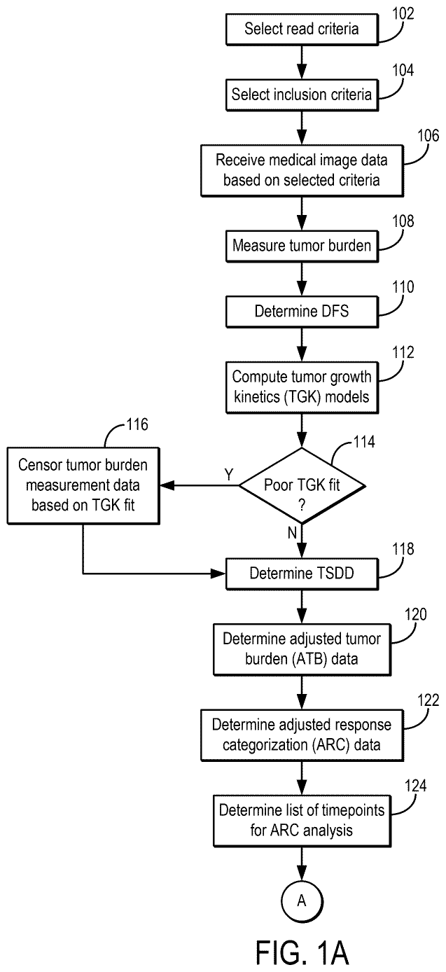

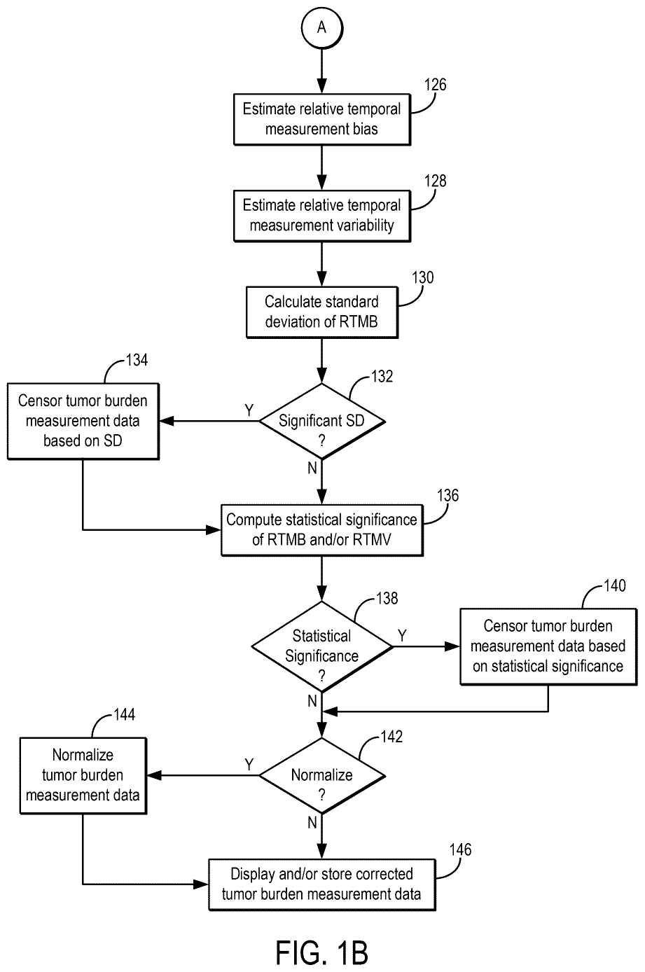

Referring now to FIGS. 1A and 1B, a flowchart illustrating the steps of an example method for evaluating and correcting for temporal measurement bias in tumor burden measurement data is illustrated.

The method includes a user selecting read criteria with a computer system, or otherwise receiving the read criteria with the computer system, as indicated at step 102. As a non-limiting example, the read criteria may be a RECIST (Response Evaluation Criteria in Solid Tumors) criteria, such as RECIST 1.1.

Using the selected read criteria, a study or dataset that meets inclusion criteria is then selected for processing, as indicated at step 104. The inclusion criteria may include the presence of solid tumors in medical images, the number of timepoints at which medical images were acquired, number of days between scans, imaging modalities used, and presence of measurable target lesions at a baseline timepoint. For instance, patients should have evaluable radiological scans containing solid tumors taken at a minimum of three unique timepoints, including baseline or C1D1. Additionally or alternatively, the number of days between scans should be known or able to be derived from the available scan dates. A single modality should be consistently used across all timepoints included in the analysis, and patients should also have at least one measurable target lesion at the baseline timepoint.

Medical image data associated with the selected study or dataset are then received with the computer system, as indicated at step 106. Receiving the medical image data may include retrieving such data from a memory or other suitable data storage device or medium. Additionally or alternatively, receiving the medical image data may include acquiring such data with a medical imaging system and transferring or otherwise communicating the data to the computer system, which may be a part of the medical imaging system.

The medical image data may include medical images of one or more patients that have been acquired using one or more imaging modalities. For instance, the medical image data may include magnetic resonance images acquired with a magnetic resonance imaging (MRI) system, computed tomography (CT) images acquired with a CT system, x-ray images acquired with an x-ray imaging system (e.g., a fluoroscopy or C-arm system), ultrasound images acquired with an ultrasound imaging system, positron emission tomography (PET) images acquired with a PET system, single photon emission computed tomography (SPECT) images acquired with a SPECT system, or the like.

The medical image data can include images acquired from a plurality of timepoints. The plurality of timepoints can correspond to different hours, days, weeks, months, years, etc., when the patient was imaged with one or more of the imaging modalities. For instance, each set of medical image data received by the computer system can be associated with a date (e.g., a particular day). Preferably, the medical image data will include medical images acquired at different timepoints using the same modality or modalities, such that medical images from the same imaging modality can be analyzed with respect to time.

The tumor burden for the subject is then measured, calculated, or otherwise estimated from the medical image data, as indicated at step 108. By way of example, one or more read criteria can be used when measuring tumor burden at each timepoint at which medical images were acquired. The tumor burden measure may include tumor size, tumor volume, sum of diameters (SOD), product of diameters (POD), and/or total tumor volume.

In some embodiments, any suitable technique or combination of techniques can be used to determine tumor burden. For example, a machine learning model can be used to calculate a volume from the medical image data and/or assist a user (e.g., a radiologist, an oncologist, etc.) to calculate a volume from the medical image data. As another example, a radiologist can draw (e.g., using input(s) of the computer system) a region of interest (ROI) on multiple 2D slices of the medical image data indicative of an outline of the tumor in that slice of image. In such an example, radiology software can use positional information from the slices and the outline drawn by the radiologist (e.g., using positional DICOM metadata) to compute a volume of the tumor from the drawn ROIs.

The days from start (DFS) for each timepoint is also determined, as indicated at step 110. The start timepoint can be defined as the baseline scan date for the subject, C1D1, or another suitable initial or reference timepoint. As a non-limiting example, DFS can be calculated as the time from the initiation of imaging and/or treatment using the following formula:

$$DFS = {t}_{i} - {t}_{0}$$

where tᵢ is the timepoint date, and t₀ is the start date.

One or more TGK models are then computed or otherwise constructed, as indicated at step 112. By way of example, the TGK model may be an eTGK model, as described above. The eTGK models can be computed by using a curve fitting algorithm. As a non-limiting example, the following formula can be used for the curve fitting algorithm:

$$Tumor burden = {e}^{- dt} + {e}^{gt} - 1 ; \qquad ( 1 )$$

where t is time, d is a decay constant and g is a growth constant. As another example, the following tumor growth model can be used:

$$Tumor burden = α {e}^{- dt} + ( 1 - α ) {e}^{gt} - 1 , ; \qquad ( 2 )$$

where α=[0, 1], and represents a proportion of tumor cells that respond to treatment. In some embodiments, any suitable curve fitting algorithm can be used to find values for d and g, based on the at least three tumor burden measurements (e.g., tumor size, tumor volume, SOD, POD, etc.) and Eqns. (1) or (2). For example, the scipy.optimize.curve_fit Python function can be used fit a curve based on the tumor burden measurements and Eqns. (1) or (2).

As indicated at decision block 114, subjects whose eTGK models have poor fits can be censored at step 116. In this way, the tumor burden measurements associated with those subjects may be removed from the tumor burden measurement data at step 116, while the method proceeds for other data that are not censored. A poor fit can include models with a goodness of fit metric that is below a certain threshold. For example, the threshold can be an R² value that is less than 0.5.

The temporal measurement bias of the remaining tumor burden measurements are then determined by generally identifying a median scan date, or other reference timepoint, and comparing measured tumor burden at each timepoint relative to this reference timepoint.

For each timepoint, the timepoint scan date distribution (TSDD) is determined, as indicated at step 118. The TSDD represents a density of DFS measurements for a defined timepoint, as determined by on-study conduct. By way of example, the TSDD can be determined by noting the earliest observed DFS, the median observed DFS, and the latest observed DFS for the collected dataset. The median DFS may also be referred to as the timepoint center of window (TCW). The TCW can represent the intended DFS that visitation is scheduled to occur, as defined by the study protocol or the median DFS measured at a given timepoint.

For each day in the range between the earliest and latest days, the adjusted tumor burden (ATB) is determined for each patient, as indicated at step 120. In general, the ATB represents a theoretical measurement of tumor burden based on the patient eTGK model. By way of example, the ATB can be calculated using the following formula:

$$ATB ( t ) = {e}^{- dt} + {e}^{gt} - 1 ; \qquad ( 3 )$$

where d is the decay constant, g the growth constant, and t is the adjusted DFS. The ATB can be determined for each patient using their corresponding eTGK constants. For example, ATB(30)=e^{-d*30}+e^{g*30}−1, where d and g are the patient constants from eTGK and the day calculated is 30 DFS.

Using the ATB values generated in the previous step, the selected read criteria are applied and the adjusted response categorization (ARC) for each patient is determined, as indicated at step 122. The ARC represents the measurement of response based on the ATBs generated by patient eTGK models according to user selected read criteria.

Using the TSDD, a list of times to use when analyzing shifts in ARCs is determined at step 124. As an example, the list of times can be selected as times corresponding to the 2.5^{th}, 25^{th}, 50^{th}, 75^{th}, and/or 97.5^{th} percentile DFS for each TSDD. Additionally or alternatively, every day within the TSDD can be used to measure daily changes in response.

The relative temporal measurement bias (RTMB) is then measured for each ATB, as indicated at step 126. The RTMB represents a percent difference between ATB values when compared against a reference point. As a non-limiting example, the reference point can be the TCW as determined above. RTMB can be calculated using the following formula:

$$( t , {t}_{r} ) = \frac{ATB ( t ) - ATB ( {t}_{r} )}{ATB ( {t}_{r} )} \times 100 \%$$

where t is a selected DFS, and tr is the reference point DFS (e.g., TCW or another reference timepoint). The temporal measurement bias (TMB) may also be determined as the difference between ATB values when compared against the reference point, typically the TCW, using the following formula:

$$TMB ( t , {t}_{r} ) = ATB ( t ) - ATB ( {t}_{r} )$$

where t is a selected DFS, and tr is the reference point DFS.

The relative temporal measurement variability (RTMV) can also be calculated, as indicated at step 128. In general, RTMV can be calculated by determining the percent difference of ARCs in comparison to their unadjusted response categorization (URC). The URC represents an on-study measurement of response based on unadjusted tumor burden (UTB) according to the selected read criteria. In general, the UTB represents the radiologist or platform-assisted measurement of tumor burden based on segmentations produced by on-study conduct.

The RTMV provides a relative measure (e.g., the percent difference) of the extent to which temporal categorizations differ from each other over a defined period, indicating the consistency of these measurements. Because RTMV is based, at least in part, on the response criteria categorizations available, the calculations for RTMV will be dependent on the selected read criteria. As one non-limiting example, RTMV can be calculated using the following formulas when the selected read criteria correspond to the RECIST 1.1 criteria:

$${RTMV}_{PR} = ( \frac{{n}_{APR} - {n}_{UPR}}{{n}_{UPR}} ) \times 100 \%$$

$${RTMV}_{SD} = ( \frac{{n}_{ASD} - {n}_{USD}}{{n}_{USD}} ) \times 100 \%$$

$${RTMV}_{PD} = ( \frac{{n}_{APD} - {n}_{UPD}}{{n}_{UPD}} ) \times 100 \%$$

where n_{UPR}, n_{USD} and n_{UPD} represent the count of URC partial response (PR), stable disease (SD), and progressive disease (PD) respectively. Similarly, n_{APR}, n_{ASD} and n_{APD} represent the count of ARC PR, SD, and PD from a theoretical reference point. In other implementations, RTMV can be calculated for additional response categorizations (e.g., complete response (CR)) or alternatively for fewer response categorizations that those listed above.

When the selected read criteria is different from RECIST 1.1, the formulas for calculating RTMV will change based on the available response criteria categorizations. For instance, if the selected read criteria are Response Assessment in Pediatric Neuro-Oncology (RAPNO) criteria, the calculations may include calculating RTMV for different response categorizations, such as minor response, major response, etc.

The temporal measurement variability (TMV) can also be calculated. The TMV represents a measure of the extent to which temporal categorizations differ from each other over a defined period, indicating the consistency of these measurements, and can be calculated using the following formulas:

$${TMV}_{PR} = ( {n}_{APR} - {n}_{UPR} ) ,$$

$${TMV}_{SD} = ( {n}_{ASD} - {n}_{USD} ) ,$$

$${TMV}_{PD} = ( {n}_{APD} - {n}_{UPD} )$$

Where n_{UPR}, n_{USD} and n_{UPD} represent the count of URC PR, SD, and PD respectively. Similarly, n_{APR}, n_{ASD} and n_{APD} represent the count of ARC PR, SD, and PD from a theoretical reference point. Like the RTMV calculations, the TMV calculations will be dependent on the selected read criteria. Accordingly, additional or alternative TMV calculations may be performed based on the available and desired response criteria categorizations.

The standard deviation of the RTMBs can then be calculated, as indicated at step 130. Based on these values, patients can be censored from the study as determined at decision block 132. For example, if a patient exists outside a ±3 standard deviation range of RTMBs, they can be censored from the study at step 134. In this way, patients who are identified as changing a lot (e.g., have tumor growth curves with a lot of change) can be censored out of the study dataset. For example, the tumor burden measurement data associated with these subjects can be removed from the tumor burden measurement data at step 134 while the method proceeds on the remaining tumor burden measurement data. Additionally, patients who are identified as outliers, but not changing a lot, can remain in the study dataset.

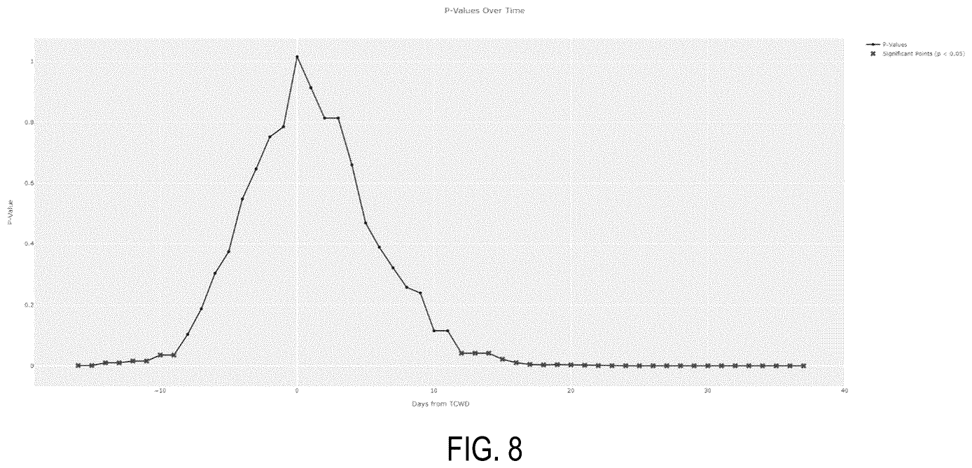

The statistical significance and effect size of the RTMB and RTMV between each selected adjusted DFS can be determined against the UTBs and URCs, respectively, as indicated at step 136. If a statistical significance is identified at decision block 136, then a decision can be made at decision block 138 whether to additionally censor the tumor burden measurement data based on the computed statistical significance. For example, with reference to FIG. 8 as a non-limiting example, if the computed p-value over time indicates a statistically significant deviation from the reference point, then the tumor burden measurement data can be identified as from a scan date at which there has been too much change in the tumor to be reliably included in subsequent analyses. In this way, patients who are identified as changing a lot can be censored out of the study dataset. For example, the tumor burden measurement data associated with these subjects can be removed from the tumor burden measurement data at step 140 while the method proceeds on the remaining tumor burden measurement data.

Additionally or alternatively, a decision is made at decision block 142 whether to normalize the tumor burden measurement data. This decision can be made based at least in part on the statistical significance of RTMB and/or RTMV calculated in step 136. In other examples, data normalization can be performed based on other criteria, including a user selecting that data normalization should be performed. Tumor burden measurement data that are selected for data normalization may then be normalized at step 144. As a non-limiting example, data normalization can be performed by choosing an insignificant DFS (e.g., TCW) and replacing each patient's URCs with the corresponding ARCs from that DFS point. Additionally or alternatively, data normalization can be performed relative to other timepoints. In this way, the measurements, responses, or other data can be normalized to represent what they would be like at different timepoints, such as day 30, day 50, etc.

Based on the measured temporal bias, censored data, and other adjusted data (e.g., normalized data), a corrected tumor burden measurement dataset is formed for the study. This corrected dataset can then be stored for later use or processing, as indicated at step 146. In general, the correct data set is processed as described above to remove or otherwise reduce the effects of temporal measurement bias in tumor burden and/or response measurements.

As one non-limiting example, the adjusted dataset can be used to generate a report that is displayed to a user via the computer system. For example, in addition to above, the disclosed methods can be used to calculate multiple metrics and figures to assist in data analysis, including timepoint table summaries; ARC and URC counts and proportions; FE-test statistic and p-value for TCW versus day adjusted response sates; t-test for difference of means between treatment groups (e.g., HER2±); days to progression; growth constants; decay constants; response counts; DFS; and so on. Additionally or alternatively, the report may include a timepoint DFS distribution ridgeline plot, a timepoint subgroup DFS distribution ridgeline plot, a response count comparison line plot, a response count subgroup comparison line plot, a PR/PD response ratio line plot, a PR/PD subgroup response ratio line plot, a significance adjusted response measurements from TCW plot; and/or a relative bias between adjusted SOD measurements and median day plot.

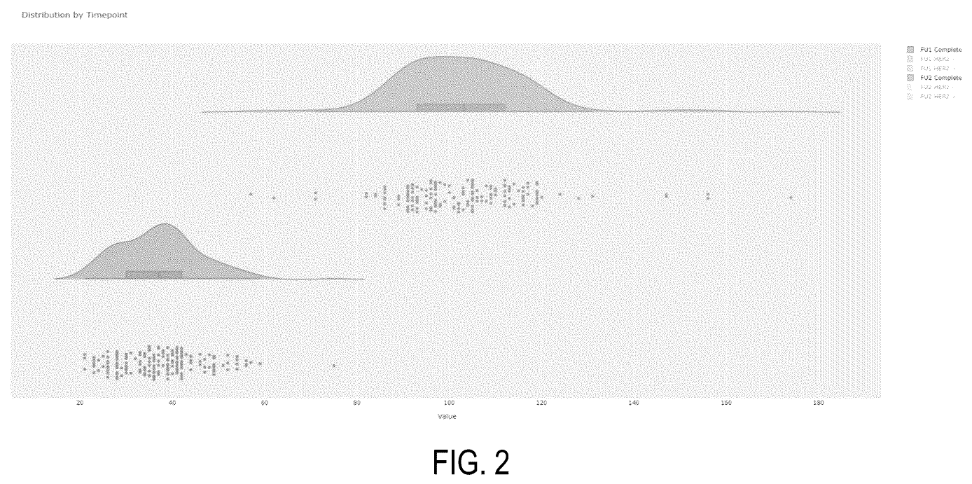

By way of example, FIG. 2 illustrates an example timepoint DFS distribution ridgeline plot. As shown in FIG. 2, the blue ridgeline displays the density of DFS for Follow-up 1 and the purple ridgeline displays the density of DFS for Follow-up 2. The scatter dots below each ridgeline represent each data point measured at each Follow-up. This figure illustrates the distribution of timepoints based on DFS with the days from the start sorted along the horizontal axis. Each ridgeline represents the density of DFS measurements at different timepoints throughout the study duration.

Advantageously, the timepoint DFS distribution ridgeline plot provides a visual representation of how DFS measurements are distributed over time during the study period. This plot helps identify clusters or patterns in DFS measurements at specific timepoints, indicating potential critical periods or milestones in disease progression or response to treatment. These data can be interpreted to identify peaks or clusters in the ridgelines, which suggest periods of frequent DFS measurements. Wider distributions also indicate that the timing of patient imaging is more sporadic and more likely to contain temporal measurement bias. Ideally, the ridgelines are separated by sharp peaks and short tails. The skewness or tilt of the distribution can indicate whether patients tended to be imaged earlier, or later on average. Areas of overlap in the plot may indicate the presence of unscheduled visits, or that visitation windows and imaging frequency are not balanced.

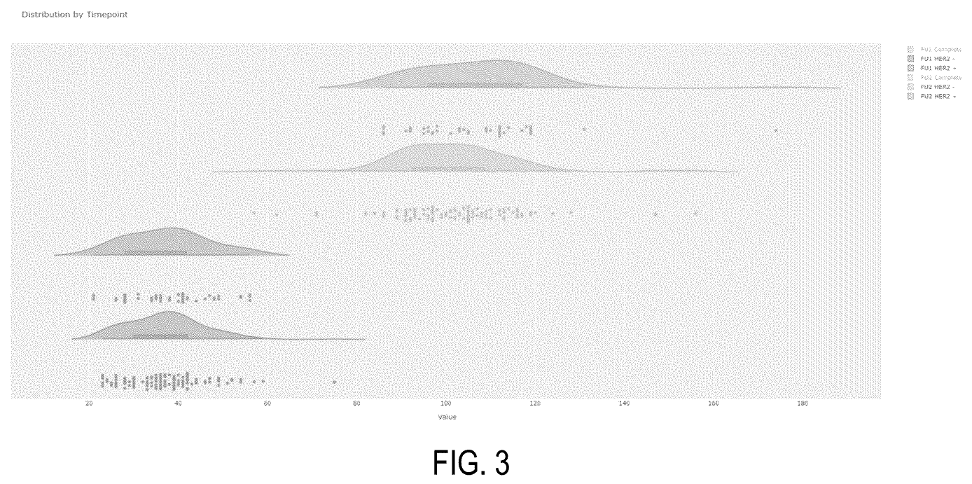

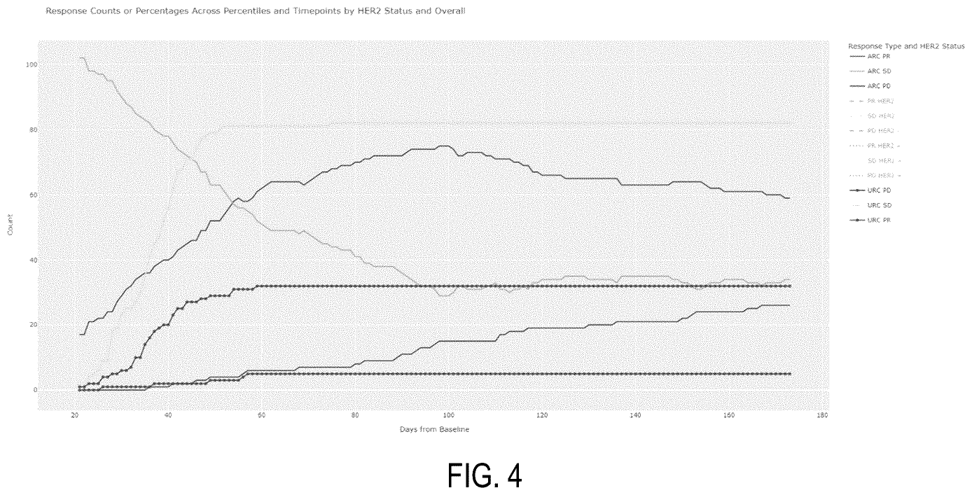

By way of example, FIG. 3 illustrates an example timepoint subgroup DFS distribution ridgeline plot. As shown in FIG. 3, the red ridgeline displays the density of HER2−DFS for Follow-up 1 and the green ridgeline displays the density of HER2+DFS for Follow-up 1. The orange ridgeline displays the density of HER2−DFS for Follow-up 2 and the teal ridgeline displays the density of HER2+DFS for Follow-up 2. The scatter dots below each ridgeline represent each data point measured at each Follow-up. This figure illustrates the distribution of subgroups based on DFS with the days from the start sorted along the horizontal axis. Each subgroup ridgeline represents the density of DFS measurements at different timepoints throughout the study duration.

Advantageously, the timepoint subgroup DFS distribution ridgeline plot provides a visual representation of how subgroup DFS measurements are distributed over time during the study period. This can help identify differences in clusters or patterns of subgroup DFS measurements at specific timepoints, indicating potential TMV due to uneven imaging windows. For example, the timepoint subgroup DFS distribution ridgeline plot can be analyzed to identify features similar to those in the standard ridgeline plot interpretations above. Additionally, the subgroup comparison allows for the identification of patients that should be censored when the tails of the distributions are not aligned, as well as the identification of potential temporal measurement variability by looking for misalignment between the shape and position of each subgroup's ridgelines.

By way of example, FIG. 4, illustrates an example response count comparison line plot. As shown in FIG. 4, the solid red, green, and yellow lines display the count of eTGK ARCs for PD, PR, and SD respectively. Only one response is counted per patient per day. The dotted red, green, and yellow lines display the cumulative count of URC for PD, PR, and SD respectively. Cumulative response counts are updated each time a unique patient's URC is measured, and only one response is counted per subject with a preference for the most recent available response categorization.

Advantageously, the response count comparison line plot illustrates the dynamic changes in RECIST 1.1 response counts over time, incorporating both dataset-wide ARCs and cumulative URCs. This plot facilitates the identification of critical timepoints or intervals where imaging may be essential for accurately assessing treatment response. Additionally, this plot enables subgroup comparisons, allowing for the examination of response rate disparities between different study cohorts on a day-to-day basis.

The effectiveness of treatment interventions or disease progression can be indicated by monitoring the trend of response categorizations over time. Periods of sharp slopes are not as ideal for capturing less temporal measurement variability as periods of flat changes in response rates, or dips on the curves. Periods of steady or sudden increases in theoretical response categorization slopes can suggest that scans conducted before or during the period may fail to capture changes in disease status. For example, a follow-up visitation scheduled for day 80 would fail to capture the changes in response from SD categorization into PR and PD cases that would have been otherwise observed had the visitation been scheduled for day 100 instead. In this example the range from day 80 to day 100 shows evidence of temporal measurement variability.

The response count comparison line plot also provides valuable insights into the temporal dynamics of treatment responses, guiding clinical decision-making and research strategies. For instance, instead of limiting response categorization measurements to the dates of imaging, the theoretical eTGK responses are able to display the day-to-day expected fluctuations in response categorizations including those that would be otherwise outside the protocol defined imaging windows for follow-up scheduling.

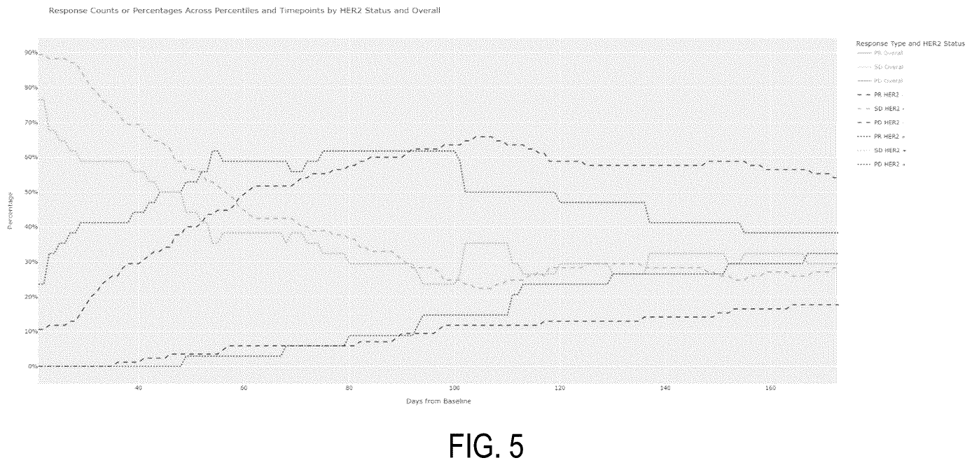

By way of example, FIG. 5 illustrates an example response count subgroup comparison line plot. In FIG. 5, the dashed red, green, and yellow lines display the HER2− proportion of eTGK ARCs for PD, PR, and SD respectively. The dotted red, green, and yellow lines display the HER2+proportion of eTGK URCs for PD, PR, and SD respectively. Only one response is counted per patient per day for each proportion.

Advantageously, the response count subgroup comparison line plot illustrates the dynamic changes in subgroup RECIST 1.1 response proportions over time and incorporated adjusted measurements. The response count subgroup comparison line plot facilitates the identification of critical intervals where one treatment or patient subgroup may be performing differently than the other. This plot supports the hypothesis that treatment responses may vary with time, highlighting potential biases if imaging occurs before response fluctuations. The response count subgroup comparison line plot also enables subgroup comparisons, allowing for the examination of response rate disparities between different study cohorts on a day-to-day basis.

In addition to the standard response count plot interpretations above, the subgroup comparison allows for inspecting differences in the magnitude of projected response rates between cohorts. For example, the figure above displays that the HER2+cohort begins to deviate with higher rates of PD in comparison to the HER2−cohort after day 60. By day 120 there are almost twice as many PDs in the HER2+cohort, proportionally. Depending on when imaging is taken for the second follow-up, there may be significant differences in response rates, signifying temporal measurement bias.

Additionally or alternatively, the response count subgroup comparison line plot enables inspecting trends and potential temporal measurement bias in subgroup response rates across the study duration. For example, the HER2+cohort appears to be responding better to treatment during the initial stages of the study. Prior to day 100, there are proportionally more HER2+PRs than in HER2−; if the second follow-up had been scheduled for at or before day 100, the study would struggle to yield a significant difference in rates in comparison to 10 days before or after.

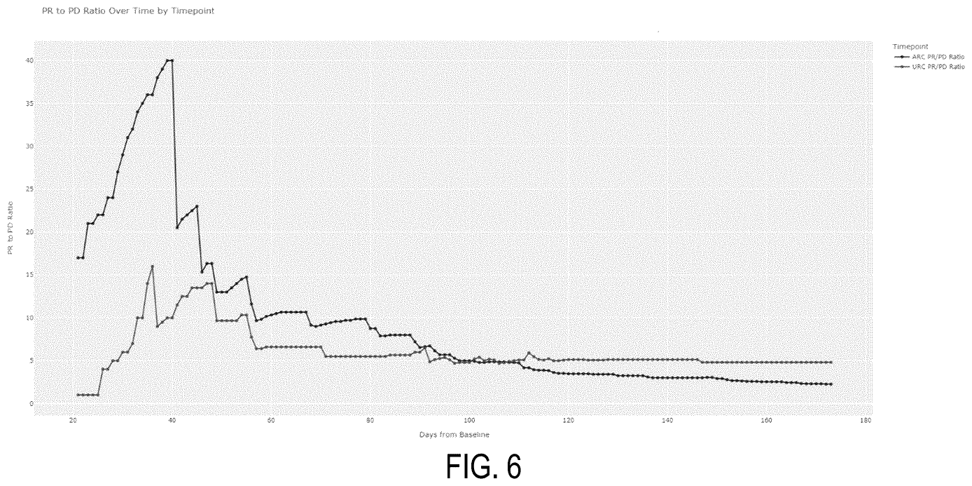

By way of example, FIG. 6 illustrates an example PR/PD response ratio line plot. In FIG. 6, the blue line represents the theoretical Adjusted PR/PD ratios. It displays the dataset wide adjusted ratio of Partial Response to Progressive Disease categorization counts. Only one response is counted per patient.

The red line above displays the cumulative ratio of PR (partial response) to PD (progressive disease) categorization counts. Cumulative response counts are updated each time a unique patient's URC is measured, and only one response is counted per subject with a preference for the most recent available response categorization.

The PR/PD response ratio line plot provides insights into the balance between Partial Response (PR) and Progressive Disease (PD) categorizations over the duration of the study. In this way, the PR/PD response ratio line plot can help evaluate the trend of treatment response by examining how the ratio of PR to PD categorizations changes over time.

For example, fluctuations in the ratio indicate shifts in the balance between PR and PD responses, thereby reflecting changes in treatment efficacy or disease progression. Periods of sharp changes are less ideal as imaging dates, as patients are still settling into a response categorization signifying noticeable TMV. On the other hand, periods of stability in the blue line are more ideal as imaging dates, as patients are not expected to change response categorizations signifying little to no TMV. A rising ratio suggests an increasing proportion of PR categorizations relative to PD categorizations, indicating potential treatment effectiveness or disease control. Conversely, a declining ratio may signal a higher prevalence of PD categorizations compared to PR categorizations, suggesting potential treatment resistance or disease advancement.

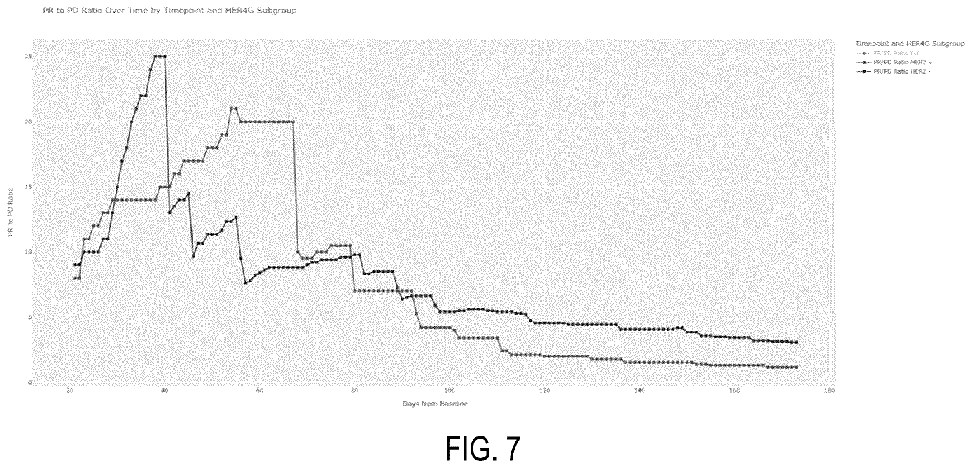

By way of example, FIG. 7 illustrates an example PR/PD subgroup response ratio line plot. In FIG. 7, the blue and red lines represent the ratios for the HER2− and HER2+treatment groups, respectively. The PR/PD subgroup response ratio line plot displays the dataset wide adjusted ratio of PR to PD categorization counts. Only one response is counted per patient.

Advantageously, the PR/PD subgroup response ratio line plot provides insights into the balance between PR and PD categorizations over the duration of the study, emphasizing differences between patient cohorts. This plot evaluates the trend of treatment response by examining how the ratio of PR to PD categorizations changes over time, with separate lines delineating subgroups for comparison.

Fluctuations in the ratios of each subgroup indicate shifts in the balance between response rates, reflecting changes in treatment efficacy or disease progression within each subgroup. The blue line above indicates that after day 80 the HER2− cohort begins performing better, in terms of ratio of PRs to PDs. A follow-up visitation scheduled for day 100 is able to display a more pronounced distinction in treatment efficacy than day 80, displaying a potential TMV. Periods where PR/PD ratios between subgroups overlap or change direction may not be ideal imaging times for cohort comparison purposes.

By way of example, FIG. 8 illustrates an example significance adjusted response measurements from TCW plot; and/or a relative bias between adjusted SOD measurements and median day plot. FIG. 8 compares the ARCs at each day against the TCW. The x-axis represents the relative days from TCW, and the y-axis represents the statistical significance metric of choice (e.g., the p-value associated with G-tests). The red markers overlayed on the observations signify statistically significant deviations representing temporal measurement bias. For example, a red marker is overlayed on the point-9 days from TCW above (Day 28) because the significance value is 0.025 which is less than the alpha set at 0.05.

Advantageously, the significance adjusted response measurements from TCW plot evaluates the significance values of the theoretical ARC rates to visualize when the ARCs significantly differ from the TCW and temporal measurement bias begins to impact the reliability of interpatient comparisons. The significance adjusted response measurements from TCW plot provides insights into the appropriateness of the measurement used on study and whether normalization should occur. For example, patients imaged on DFS that significant differ from TCW in response categorizations, could be identified and then their measurements normalized to the TCW to align more with the rest of the cohort.

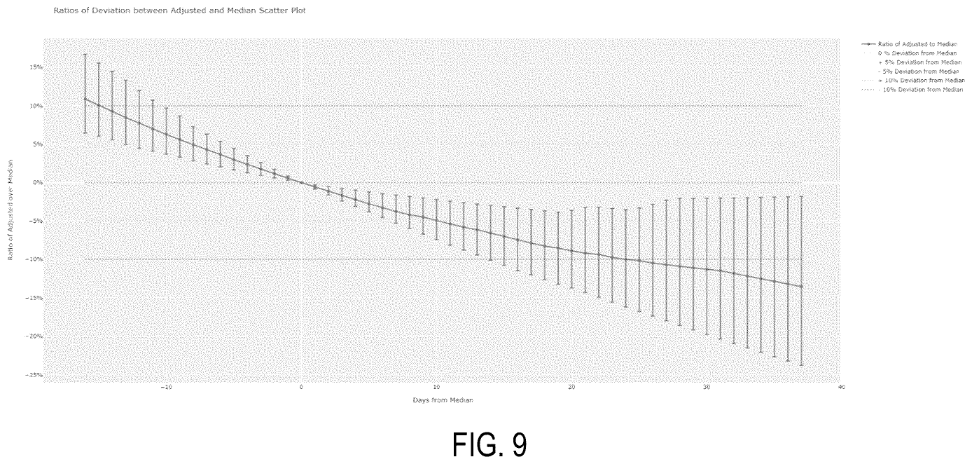

By way of example, FIG. 9 illustrates an example relative bias between adjusted SOD measurements and median day plot. FIG. 9 compares the ratio between the adjusted SOD measurements at each day against the TCW SOD. The x-axis represents the relative days from TCW, and the y-axis represents the ratio between adjusted days and the TCW day SOD values. The yellow markers overlayed on the observations signify ±5% ratios, representing minimal RTMB, while the red ones signify ±10% ratios representing greater RTMB. Other thresholds may be more appropriate for different datasets, particularly if there is a biological rationale for such a threshold. The labeled points for each DFS shown are the 25^{th}, 50^{th}, and 75^{th} percentile (bottom, middle, and top points respectively) RTMB from TCW across all patients. The median (e.g., 50^{th} percentile) RTMBs are connected to display the directionality of temporal bias.

Advantageously, the relative bias between adjusted SOD measurements and median day plot evaluates the RTMB to determine when the ATBs significantly differ from the TCW. When the 50^{th} percentile points occur outside these boundaries, then more than 50% of patients exhibit RMTB. The 25^{th} and 75^{th} percentile RTMB display the interpatient variability of temporal bias with respect to ATB.

The relative bias between adjusted SOD measurements and median day plot provides insights into the appropriateness of the tumor burden values used on study and whether normalization should occur for certain patients. For example, more than 50% of patients imaged 25 days after the TCW exhibit significant RTMB. Therefore, it would be appropriate to normalize patients who were measured/imaged at or later than 25 days after the TCW to adjust for a fairer comparison.

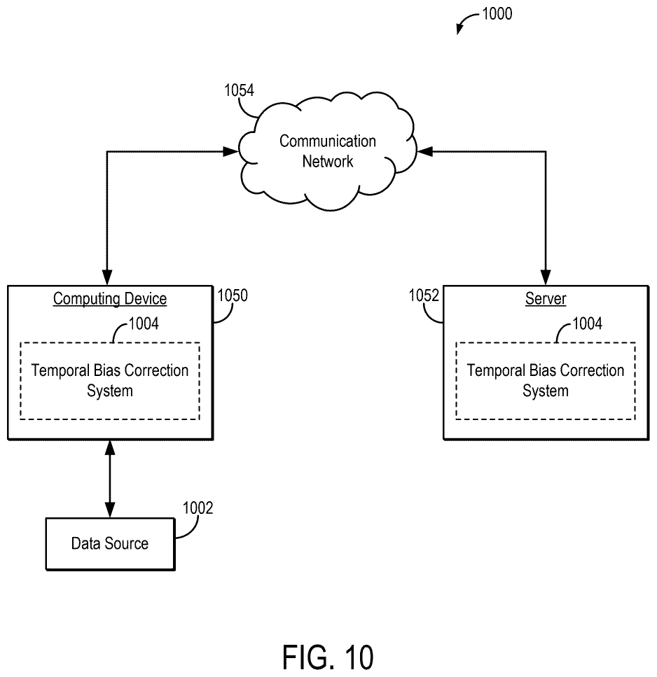

FIG. 10 shows an example of a system 1000 for correcting or otherwise reducing temporal measurement bias and/or variability in tumor measurements in accordance with some embodiments described in the present disclosure. As shown in FIG. 10, a computing device 1050 can receive one or more types of data (e.g., medical image data, read criteria) from data source 1002. In some embodiments, computing device 1050 can execute at least a portion of a tumor measurement temporal bias correction system 1004 to correct or otherwise reduce temporal measurement bias and/or variability in tumor measurements based on data received from the data source 1002.

Additionally or alternatively, in some embodiments, the computing device 1050 can communicate information about data received from the data source 1002 to a server 1052 over a communication network 1054, which can execute at least a portion of the tumor measurement temporal bias correction system 1004. In such embodiments, the server 1052 can return information to the computing device 1050 (and/or any other suitable computing device) indicative of an output of the tumor measurement temporal bias correction system 1004.

In some embodiments, computing device 1050 and/or server 1052 can be any suitable computing device or combination of devices, such as a desktop computer, a laptop computer, a smartphone, a tablet computer, a wearable computer, a server computer, a virtual machine being executed by a physical computing device, and so on. The computing device 1050 and/or server 1052 can also reconstruct images from the data.

In some embodiments, data source 1002 can be any suitable source of data (e.g., measurement data, images reconstructed from measurement data, processed image data), such as a medical imaging system (e.g., an MRI system, a CT system, an ultrasound system, a PET system, a SPECT system, etc.), another computing device (e.g., a server storing measurement data, images reconstructed from measurement data, processed image data), and so on. In some embodiments, data source 1002 can be local to computing device 1050. For example, data source 1002 can be incorporated with computing device 1050 (e.g., computing device 1050 can be configured as part of a device for measuring, recording, estimating, acquiring, or otherwise collecting or storing data). As another example, data source 1002 can be connected to computing device 1050 by a cable, a direct wireless link, and so on. Additionally or alternatively, in some embodiments, data source 1002 can be located locally and/or remotely from computing device 1050, and can communicate data to computing device 1050 (and/or server 1052) via a communication network (e.g., communication network 1054).

In some embodiments, communication network 1054 can be any suitable communication network or combination of communication networks. For example, communication network 1054 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 1054 can be a local area network, a wide area network, a public network (e.g., the Internet), a private or semi-private network (e.g., a corporate or university intranet), any other suitable type of network, or any suitable combination of networks. Communications links shown in FIG. 10 can each be any suitable communications link or combination of communications links, such as wired links, fiber optic links, Wi-Fi links, Bluetooth links, cellular links, and so on.

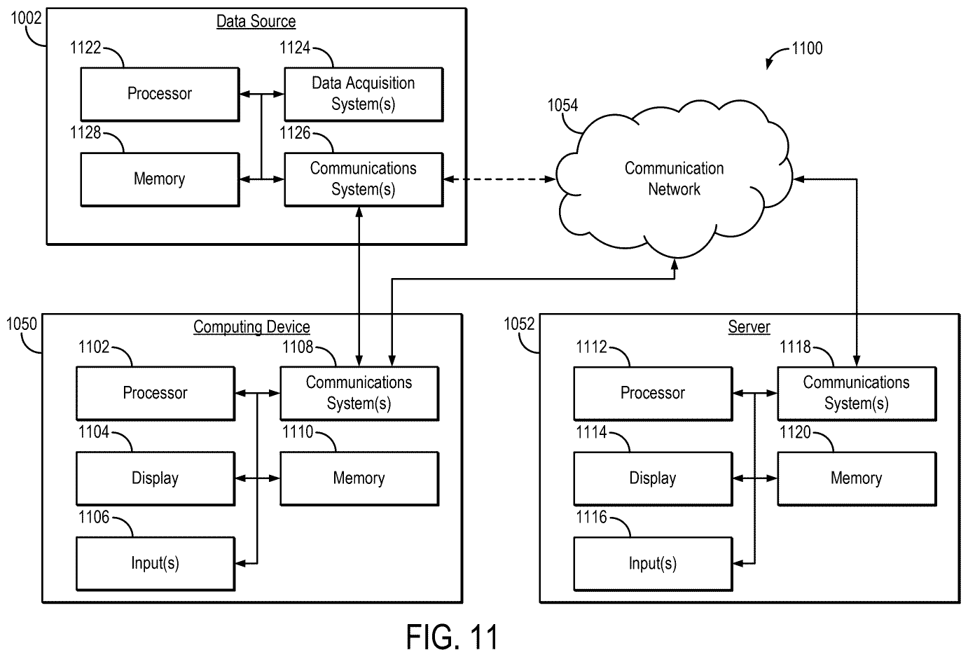

Referring now to FIG. 11, an example of hardware 1100 that can be used to implement data source 1002, computing device 1050, and server 1052 in accordance with some embodiments of the systems and methods described in the present disclosure is shown.

As shown in FIG. 11, in some embodiments, computing device 1050 can include a processor 1102, a display 1104, one or more inputs 1106, one or more communication systems 1108, and/or memory 1110. In some embodiments, processor 1102 can be any suitable hardware processor or combination of processors, such as a central processing unit (CPU), a graphics processing unit (GPU), and so on. In some embodiments, display 1104 can include any suitable display devices, such as a liquid crystal display (LCD) screen, a light-emitting diode (LED) display, an organic LED (OLED) display, an electrophoretic display (e.g., an “e-ink” display), a computer monitor, a touchscreen, a television, and so on. In some embodiments, inputs 1106 can include any suitable input devices and/or sensors that can be used to receive user input, such as a keyboard, a mouse, a touchscreen, a microphone, and so on.

In some embodiments, communications systems 1108 can include any suitable hardware, firmware, and/or software for communicating information over communication network 1054 and/or any other suitable communication networks. For example, communications systems 1108 can include one or more transceivers, one or more communication chips and/or chip sets, and so on. In a more particular example, communications systems 1108 can include hardware, firmware, and/or software that can be used to establish a Wi-Fi connection, a Bluetooth connection, a cellular connection, an Ethernet connection, and so on.

In some embodiments, memory 1110 can include any suitable storage device or devices that can be used to store instructions, values, data, or the like, that can be used, for example, by processor 1102 to present content using display 1104, to communicate with server 1052 via communications system(s) 1108, and so on. Memory 1110 can include any suitable volatile memory, non-volatile memory, storage, or any suitable combination thereof. For example, memory 1110 can include random-access memory (RAM), read-only memory (ROM), electrically programmable ROM (EPROM), electrically erasable ROM (EEPROM), other forms of volatile memory, other forms of non-volatile memory, one or more forms of semi-volatile memory, one or more flash drives, one or more hard disks, one or more solid state drives, one or more optical drives, and so on. In some embodiments, memory 1110 can have encoded thereon, or otherwise stored therein, a computer program for controlling operation of computing device 1050. In such embodiments, processor 1102 can execute at least a portion of the computer program to present content (e.g., images, user interfaces, graphics, tables), receive content from server 1052, transmit information to server 1052, and so on. For example, the processor 1102 and the memory 1110 can be configured to perform the methods described herein (e.g., the method of FIG. 1).

In some embodiments, server 1052 can include a processor 1112, a display 1114, one or more inputs 1116, one or more communications systems 1118, and/or memory 1120. In some embodiments, processor 1112 can be any suitable hardware processor or combination of processors, such as a CPU, a GPU, and so on. In some embodiments, display 1114 can include any suitable display devices, such as an LCD screen, LED display, OLED display, electrophoretic display, a computer monitor, a touchscreen, a television, and so on. In some embodiments, inputs 1116 can include any suitable input devices and/or sensors that can be used to receive user input, such as a keyboard, a mouse, a touchscreen, a microphone, and so on.

In some embodiments, communications systems 1118 can include any suitable hardware, firmware, and/or software for communicating information over communication network 1054 and/or any other suitable communication networks. For example, communications systems 1118 can include one or more transceivers, one or more communication chips and/or chip sets, and so on. In a more particular example, communications systems 1118 can include hardware, firmware, and/or software that can be used to establish a Wi-Fi connection, a Bluetooth connection, a cellular connection, an Ethernet connection, and so on.

In some embodiments, memory 1120 can include any suitable storage device or devices that can be used to store instructions, values, data, or the like, that can be used, for example, by processor 1112 to present content using display 1114, to communicate with one or more computing devices 1050, and so on. Memory 1120 can include any suitable volatile memory, non-volatile memory, storage, or any suitable combination thereof. For example, memory 1120 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1120 can have encoded thereon a server program for controlling operation of server 1052. In such embodiments, processor 1112 can execute at least a portion of the server program to transmit information and/or content (e.g., data, images, a user interface) to one or more computing devices 1050, receive information and/or content from one or more computing devices 1050, receive instructions from one or more devices (e.g., a personal computer, a laptop computer, a tablet computer, a smartphone), and so on.

In some embodiments, the server 1052 is configured to perform the methods described in the present disclosure. For example, the processor 1112 and memory 1120 can be configured to perform the methods described herein (e.g., the method of FIG. 1).

In some embodiments, data source 1002 can include a processor 1122, one or more data acquisition systems 1124, one or more communications systems 1126, and/or memory 1128. In some embodiments, processor 1122 can be any suitable hardware processor or combination of processors, such as a CPU, a GPU, and so on. In some embodiments, the one or more data acquisition systems 1124 are generally configured to acquire data, images, or both, and can include an MRI system, a CT system, or any other suitable medical imaging system. Additionally or alternatively, in some embodiments, the one or more data acquisition systems 1124 can include any suitable hardware, firmware, and/or software for coupling to and/or controlling operations of a medical imaging system (e.g., an MRI system, a CT system, etc.). In some embodiments, one or more portions of the data acquisition system(s) 1124 can be removable and/or replaceable.

Note that, although not shown, data source 1002 can include any suitable inputs and/or outputs. For example, data source 1002 can include input devices and/or sensors that can be used to receive user input, such as a keyboard, a mouse, a touchscreen, a microphone, a trackpad, a trackball, and so on. As another example, data source 1002 can include any suitable display devices, such as an LCD screen, an LED display, an OLED display, an electrophoretic display, a computer monitor, a touchscreen, a television, etc., one or more speakers, and so on.

In some embodiments, communications systems 1126 can include any suitable hardware, firmware, and/or software for communicating information to computing device 1050 (and, in some embodiments, over communication network 1054 and/or any other suitable communication networks). For example, communications systems 1126 can include one or more transceivers, one or more communication chips and/or chip sets, and so on. In a more particular example, communications systems 1126 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1128 can include any suitable storage device or devices that can be used to store instructions, values, data, or the like, that can be used, for example, by processor 1122 to control the one or more data acquisition systems 1124, and/or receive data from the one or more data acquisition systems 1124; to generate images from data; present content (e.g., data, images, a user interface) using a display; communicate with one or more computing devices 1050; and so on. Memory 1128 can include any suitable volatile memory, non-volatile memory, storage, or any suitable combination thereof. For example, memory 1128 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1128 can have encoded thereon, or otherwise stored therein, a program for controlling operation of data source 1002. In such embodiments, processor 1122 can execute at least a portion of the program to generate images, transmit information and/or content (e.g., data, images, a user interface) to one or more computing devices 1050, receive information and/or content from one or more computing devices 1050, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for correcting temporal bias in measurements of tumor burden based on medical image data, the method comprising:
receiving medical image data with a computer system, wherein the medical image data comprise medical images acquired at a plurality of timepoints for each of a plurality of subjects;
generating tumor burden measurement data from the medical image data using the computer system, wherein the tumor burden measurement data indicate a tumor burden estimate for each of the plurality of timepoints for each of the plurality of subjects;
computing a tumor growth kinetics (TGK) model for each of the plurality of subjects based on the tumor burden measurement data generated for the respective one of the plurality of subjects;
estimating a temporal bias for each of the plurality of subjects based on the TGK model for the respective one of the plurality of subjects; and
generating corrected tumor burden measurement data using the estimated temporal bias.

2. The method of claim 1, wherein estimating the temporal bias comprises:
estimating adjusted tumor burden measurement data for each of the plurality of subjects using the TGK model for the respective one of the plurality of subjects, wherein the adjusted tumor burden measurement data estimate a tumor burden for each of the plurality of subjects at a common reference timepoint; and
estimating the temporal bias using the adjusted tumor burden measurement data.

3. The method of claim 2, wherein the temporal bias comprises a relative temporal measurement bias calculated as a percent difference between adjusted tumor burden values compared against the reference timepoint.

4. The method of claim 2, wherein estimating the temporal bias further comprises:
estimating an adjusted response categorization (ARC) for each of the plurality of subjects using the TGK model for the respective one of the plurality of subjects, wherein the ARC indicates a measurement of response based on the adjusted tumor burden measurement data; and
estimating the temporal bias using the ARC for each subject.

5. The method of claim 4, wherein the temporal bias comprises a relative temporal measurement variability calculated as a percent difference of ARCs in comparison to their unadjusted response categorization.

6. The method of claim 1, wherein generating the corrected tumor burden measurement data using the estimated temporal bias comprises censoring tumor burden measurements from the tumor burden measurement data based on an analysis of the temporal bias.

7. The method of claim 1, wherein generating the corrected tumor burden measurement data using the estimated temporal bias comprises normalizing tumor burden measurements in the tumor burden measurement data using the estimated temporal bias.

8. The method of claim 7, wherein censoring tumor burden measurements comprises removing tumor burden measurements associated with subjects having a temporal bias outside a threshold standard deviation range.

9. The method of claim 1, wherein generating the corrected tumor burden measurement data using the estimated temporal bias comprises normalizing tumor burden measurements in the tumor burden measurement data using the estimated temporal bias.

10. The method of claim 9, wherein normalizing tumor burden measurements comprises replacing unadjusted response categorizations with adjusted response categorizations from a reference days from start point.

11. The method of claim 1, wherein the TGK model comprises an exponential tumor growth kinetics model.

12. The method of claim 11, wherein the exponential tumor growth kinetics model is computed using a curve fitting algorithm based on a formula comprising a decay constant and a growth constant.

13. The method of claim 1, further comprising censoring tumor burden measurement data associated with subjects whose TGK models have a goodness of fit metric below a threshold value.

14. The method of claim 1, wherein the tumor burden estimate comprises at least one of a tumor size, a tumor volume, a sum of diameters, a product of diameters, or a total tumor volume.

15. The method of claim 1, further comprising determining a days from start value for each of the plurality of timepoints, wherein the days from start value is calculated as a difference between a timepoint date and a start date.

16. The method of claim 1, further comprising determining a timepoint scan date distribution for each of the plurality of timepoints, wherein the timepoint scan date distribution represents a density of days from start measurements for a defined timepoint.

17. The method of claim 1, further comprising generating a report based on the corrected tumor burden measurement data, wherein the report is displayed to a user via the computer system.

18. A non-transitory computer-readable medium having stored thereon instructions that, when executed by a processor, cause the processor to:
access medical images obtained at multiple time intervals for a group of patients;
determine tumor burden estimates from the medical images for each time interval for each patient in the group;
fit a tumor growth kinetics model to the tumor burden estimates for each patient;
calculate a temporal measurement bias for each patient using the fitted tumor growth kinetics model;
produce bias-corrected tumor burden data based on the calculated temporal measurement bias, wherein the bias-corrected tumor burden data corrects for temporal measurement bias arising from non-uniform image acquisition times across the group of patients; and
generate and display a report indicating the bias-corrected tumor burden data.

19. The non-transitory computer-readable medium of claim 18, wherein the instructions further cause the processor to compute adjusted tumor burden values for each patient at a common reference time using the fitted tumor growth kinetics model, and wherein the temporal measurement bias is calculated as a difference between the adjusted tumor burden values and unadjusted tumor burden values.

20. The non-transitory computer-readable medium of claim 19, wherein the instructions further cause the processor to determine adjusted response categorizations based on the adjusted tumor burden values and calculate a temporal measurement variability as a difference between the adjusted response categorizations and unadjusted response categorizations.
